(19) United States
(12) Patent Application Publication
(10) Pub. No.: US 2026/0283521 A1
(43) Pub. Date: Sep. 24, 2026

(54) DRIVER STATE DETERMINATION DEVICE, IN-VEHICLE DEVICE, DRIVER STATE DETERMINATION SYSTEM, DRIVER STATE DETERMINATION METHOD, AND NON-TRANSITORY STORAGE MEDIUM STORING DRIVER STATE DETERMINATION PROGRAM

(71) Applicant: SUMITOMO ELECTRIC INDUSTRIES, LTD., Osaka-shi, Osaka (JP)

(72) Inventor: Rira MIYATA, Osaka-shi (JP)

(73) Assignee: SUMITOMO ELECTRIC INDUSTRIES, LTD., Osaka-shi, Osaka (JP)

(21) Appl. No.: 19/475,596

(22) PCT Filed: Apr. 8, 2024
(86) PCT No.: PCT/JP2024/014208

§ 371 Date: Oct. 15, 2025

(30) Foreign Application Priority Data
Apr. 20, 2023 (JP) 2023-069072

(51) Int. Cl. A61B 5/18; A61B 5/11; G01C 21/30

(52) U.S. Cl. CPC A61B 5/18; A61B 5/11; G01C 21/30; A61B 2503/22

(57) ABSTRACT

A driver state determination device includes: a measurement result acquisition unit that acquires at least one measurement result related to a change in position of the center of gravity of a driver of a vehicle; a travel route acquisition unit that acquires a travel route of the vehicle which corresponds to the measurement result; a reference data acquisition unit that acquires reference data which indicates a reference of the change in position of the center of gravity in a target route which is the travel route; and a determination unit that carries out a determination process for determining a state of the driver on the basis of the measurement result and the reference data.

### TECHNICAL FIELD

The present disclosure relates to a driver state determination device, an in-vehicle device, a driver state determination system, a driver state determination method, and a driver state determination program. This application claims priority based on Japanese Patent Application No. 2023-069072 filed on Apr. 20, 2023, the entire contents of which are incorporated herein by reference.

### BACKGROUND ART

Conventionally, a technique for determining a state of a driver who is driving a vehicle has been developed. For example, Patent Literature 1 (Japanese Unexamined Patent Application Publication No. 2002-8159) discloses the following technique. That is, a driver state determination device is provided with a body pressure sensor provided on a seat surface, and a computation device that calculates a center-of-gravity of a seat surface pressure of the driver applied to the seat surface by data outputted from the body pressure sensor, and determines the driving condition of the driver by comparing time-series data of a change in the center-of-gravity with a feature pattern in the driving state of the driver stored in advance.

Further, Patent Literature 2 (WO 2018/180331) discloses the following technique. That is, the driver state detection device includes an acceleration sensor attached to a vehicle, a center-of-gravity movement amount detection unit attached to a component constituting the vehicle and configured to detect a center-of-gravity movement amount of a body of a driver riding on the vehicle, and a driver condition determination unit configured to determine a state of the driver based on a magnitude of a deviation amount between an acceleration of the vehicle obtained by the acceleration sensor and the center-of-gravity movement amount of the body of the driver detected by the center-of-gravity movement amount detection unit.

### CITATION LIST

### Patent Literature

Patent Literature1: Japanese Unexamined Patent Application Publication No. 2002-8159
Patent Literature 2: WO 2018/180331

### SUMMARY OF INVENTION

A driver state determination device of the present disclosure includes a measurement result acquisition unit configured to acquire at least one measurement result regarding a change in a center-of-gravity position of a driver of a vehicle, a travel route acquisition unit configured to acquire a travel route of the vehicle, the travel route corresponding to the measurement result, a reference data acquisition unit configured to acquire reference data indicating a criterion for the change in the center-of-gravity position in a target route, the target route being the travel route, and a determination unit configured to perform determination processing of determining a state of the driver, based on the measurement result and the reference data.

One aspect of the present disclosure can be implemented not only as a driver state determination device including such a characteristic processing unit but also as a semiconductor integrated circuit that implements a part or all of the driver state determination device.

One aspect of the present disclosure can be implemented not only as an in-vehicle device including such a characteristic processing unit, but also as a method including such characteristic processing as steps, or as a program for causing a computer to execute such steps. In addition, one aspect of the present disclosure can be implemented as a semiconductor integrated circuit that implements a part or all of the in-vehicle device.

### BRIEF DESCRIPTION OF THE DRAWINGS

FIG. 1 is a diagram illustrating a configuration of a driver state determination system according to an embodiment of the present disclosure.

FIG. 2 is a diagram illustrating a configuration of an in-vehicle device according to the embodiment of the present disclosure.

FIG. 3 is a diagram illustrating an example of evaluation data created by the in-vehicle device according to the embodiment of the present disclosure.

FIG. 4 is a diagram illustrating a configuration of a server according to the embodiment of the present disclosure.

FIG. 5 is a diagram illustrating an example of a correspondence table stored in the server according to the embodiment of the present disclosure.

FIG. 6 is a diagram illustrating an example of a management table stored in the server according to the embodiment of the present disclosure.

FIG. 7 is a diagram for explaining an example of a comparison processing by the server according to the embodiment of the present disclosure;

FIG. 8 is a diagram illustrating an example of a management table after a table update processing by the server according to the embodiment of the present disclosure;

FIG. 9 is a flowchart illustrating an operation procedure when the in-vehicle device according to the embodiment of the present disclosure performs a processing of transmitting determination information.

FIG. 10 is a flowchart illustrating an operation procedure when the server according to the embodiment of the present disclosure performs a determination processing.

FIG. 11 is a flowchart illustrating an operation procedure when the server according to the embodiment of the present disclosure performs a determination processing.

FIG. 12 is a diagram illustrating an example of a sequence of processing of an in-vehicle device, a server, an operation management device, and a navigation device in the driver state determination system according to the embodiment of the present disclosure;

FIG. 13 is a diagram for explaining a data adjustment processing by a modification of the server according to the embodiment of the present disclosure.

### DETAILED DESCRIPTION

### Problems to be Solved by Present Disclosure

There is a demand for a technique capable of improving the accuracy of determination of the state of the driver while simplifying the configuration of the vehicle, beyond the techniques described in Patent Literature 1 and Patent Literature 2.

The present disclosure has been made to solve the above-described problems, and an object thereof is to provide a driver state determination device, an in-vehicle device, a driver state determination system, a driver state determination method, and a driver state determination program capable of improving determination accuracy of a state of a driver while simplifying a configuration of a vehicle.

### Advantageous Effects of Present Disclosure

According to the present disclosure, it is possible to improve the determination accuracy of the state of the driver while simplifying the configuration of the vehicle.

First, the contents of embodiments of the present disclosure will be listed and described.

(1) A driver state determination device according to an embodiment of the present disclosure includes a measurement result acquisition unit configured to acquire at least one measurement result regarding a change in a center-of-gravity position of a driver of a vehicle, a travel route acquisition unit configured to acquire a travel route of the vehicle, the travel route corresponding to the measurement result, a reference data acquisition unit configured to acquire reference data indicating a criterion for the change in the center-of-gravity position in a target route, the target route being the travel route, and a determination unit configured to perform determination processing of determining a state of the driver, based on the measurement result and the reference data.

With such a configuration, the state of the driver can be accurately determined without performing complicated determination processing, such as determining that the state of the driver is abnormal, for example, when the similarity between the measurement result and the reference data is low, using the measurement result regarding the change in the center-of-gravity position of the driver and the reference data indicating the criterion for the change in the center-of-gravity position in the travel route corresponding to the measurement result. In addition, in the vehicle, it is possible to eliminate the need for an in-vehicle equipment for acquiring other information used for correction of the measurement result, for example, in addition to the in-vehicle equipment for acquiring the measurement result regarding the change in the center-of-gravity position of the driver. Thus, it is possible to improve the determination accuracy of the state of the driver while simplifying the configuration of the vehicle.

(2) In the above (1), the measurement result acquisition unit may be configured to acquire a target measurement result, the target measurement result being the measurement result in the target route, and the reference data may be created based on the target measurement result.

With such a configuration, it is possible to further improve the determination accuracy of the state of the driver by using more appropriate reference data created based on the measurement result regarding the change in the center-of-gravity position of the driver in the same travel route as the acquired travel route.

(3) In the above (2), the target measurement result may be the measurement result for the driver to be subjected to the determination processing, and the reference data may be created for each driver to be subjected to the determination processing.

With such a configuration, it is possible to further improve the determination accuracy of the state of the driver by using more appropriate reference data created based on the measurement result regarding the change in the center-of-gravity position of the driver to be subjected to the determination processing.

(4) In the above (2) or (3), the determination unit may be configured to perform processing of determining whether an abnormality has occurred in the driver, based on the target measurement result, the at least one measurement result may include a plurality of measurement results, the measurement result acquisition unit may be configured to acquire the plurality of measurement results including the target measurement result, and the reference data may be created based on the target measurement result and a measurement result based on which it is determined by the determination unit that no abnormality has occurred, among the plurality of measurement results.

With such a configuration, it is possible to further improve the determination accuracy of the state of the driver by using more appropriate reference data created based on the measurement result in which the state of the driver is determined is normal in the determination processing in addition to the measurement result in the same travel route as the acquired travel route.

(5) In any one of the above (1) to (4), the reference data acquisition unit may be configured to create the reference data, based on the measurement result, and the reference data acquisition unit may be configured to exclude the measurement result from source data of the reference data when the measurement result satisfies a predetermined condition.

With such a configuration, for example, when the acquired measurement result indicates a change in the center-of-gravity position of the driver in a case where an abnormal behavior is performed by the driver, the measurement result can be excluded from the source data of the reference data, and thus more appropriate reference data can be created.

(6) In any one of the above (1) to (5), the reference data may indicate a criterion for the change in the center-of-gravity position in a case where a vehicle of an identical model to the vehicle travels along the target route.

With such a configuration, the determination processing can be performed using the reference data corresponding to the vehicle of the identical model to the vehicle driven by the driver to be subjected to the determination processing, and thus the determination accuracy of the state of the driver can be further improved.

(7) In any one of the above (1) to (6), the driver state determination device may further include a storage unit configured to store correspondence information indicating a correspondence relationship between the travel route and the reference data. The reference data acquisition unit may be configured to acquire the correspondence information from the storage unit and acquire the reference data, based on the correspondence information.

With such a configuration, it is possible to easily specify the reference data used for the determination processing using the correspondence information.

(8) An in-vehicle device according to an embodiment of the present disclosure is an in-vehicle device to be mounted in a vehicle. The in-vehicle device includes a measurement information acquisition unit configured to acquire measurement information indicating a measurement result regarding a change in a center-of-gravity position of a driver of the vehicle, a route information acquisition unit configured to acquire route information indicating a travel route of the vehicle, the travel route corresponding to the measurement result, and a communication unit configured to transmit the measurement information and the route information to an external device outside the vehicle.

With such a configuration, in the external device, the state of the driver can be determined without performing complicated determination processing, such as determining that the state of the driver is abnormal, for example, when the similarity between the measurement result and the criterion data is low, using the measurement result indicated by the measurement information received from the in-vehicle device and the criterion data indicating the criterion for the change in the center-of-gravity position in the travel route indicated by the route information received from the in-vehicle device. In addition, in the vehicle, it is possible to eliminate the need for an in-vehicle equipment for acquiring other information used for correction of the measurement result, for example, in addition to the in-vehicle equipment for acquiring the measurement result regarding the change in the center-of-gravity position of the driver. Thus, it is possible to improve the determination accuracy of the state of the driver while simplifying the configuration of the vehicle.

(9) A driver state determination system according to an embodiment of the present disclosure includes an in-vehicle device to be mounted in a vehicle, and a driver state determination device, the in-vehicle device being configured to transmit measurement information and route information to the driver state determination device, the measurement information indicating a measurement result regarding a change in a center-of-gravity position of a driver of the vehicle, the route information indicating a travel route of the vehicle, the travel route corresponding to the measurement result, the driver state determination device being configured to acquire reference data indicating a criterion for the change in the center-of-gravity position in the travel route indicated by the route information received from the in-vehicle device, the driver state determination device being configured to determine a state of the driver, based on the measurement result indicated by the measurement information received from the in-vehicle device and the reference data.

With such a configuration, the state of the driver can be accurately determined without performing complicated determination processing, such as determining that the state of the driver is abnormal, for example, when the similarity between the measurement result and the reference data is low, using the measurement result regarding the change in the center-of-gravity position of the driver and the reference data indicating the criterion for the change in the center-of-gravity position in the travel route corresponding to the measurement result. In addition, in the vehicle, it is possible to eliminate the need for an in-vehicle equipment for acquiring other information used for correction of the measurement result, for example, in addition to the in-vehicle equipment for acquiring the measurement result regarding the change in the center-of-gravity position of the driver. Thus, it is possible to improve the determination accuracy of the state of the driver while simplifying the configuration of the vehicle.

(10) A driver state determination method according to an embodiment of the present disclosure is a driver state determination method in a driver state determination device. The driver state determination method includes acquiring a measurement result regarding a change in a center-of-gravity position of a driver of a vehicle, acquiring a travel route of the vehicle, the travel route corresponding to the measurement result, acquiring reference data indicating a criterion for the change in the center-of-gravity position in a target route, the target route being the travel route, and performing determination processing of determining a state of the driver, based on the measurement result and the reference data.

With such a configuration, the state of the driver can be accurately determined without performing complicated determination processing, such as determining that the state of the driver is abnormal, for example, when the similarity between the measurement result and the reference data is low, using the measurement result regarding the change in the center-of-gravity position of the driver and the reference data indicating the criterion of the change in the center-of-gravity position in the travel route corresponding to the measurement result. In addition, in the vehicle, it is possible to eliminate the need for an in-vehicle equipment for acquiring other information used for correction of the measurement result, for example, in addition to the in-vehicle equipment for acquiring the measurement result regarding the change in the center-of-gravity position of the driver. Thus, it is possible to improve the determination accuracy of the state of the driver while simplifying the configuration of the vehicle.

(11) A driver state determination method according to an embodiment of the present disclosure is a driver state determination method in an in-vehicle system including an in-vehicle device to be mounted in a vehicle and a driver state determination device.

The driver state determination method includes transmitting, by the in-vehicle device, measurement information and route information to the driver state determination device, the measurement information indicating a measurement result regarding a change in a center-of-gravity position of a driver of the vehicle, the route information indicating a travel route of the vehicle, the travel route corresponding to the measurement result, acquiring, by the driver state determination device, reference data indicating a criterion for the change in the center-of-gravity position in the travel route indicated by the route information received from the in-vehicle device, and determining, by the driver state determination device, a state of the driver, based on the measurement result indicated by the measurement information received from the in-vehicle device and the reference data.

With such a configuration, the state of the driver can be accurately determined without performing complicated determination processing, such as determining that the state of the driver is abnormal when the similarity between the measurement result and the reference data is low, using the measurement result regarding the change in the center-of-gravity position of the driver and the reference data indicating the criterion of the change in the center-of-gravity position in the travel route corresponding to the measurement result. In addition, in the vehicle, it is possible to eliminate the need for an in-vehicle equipment for acquiring other information used for correction of the measurement result, for example, in addition to the in-vehicle equipment for acquiring the measurement result regarding the change in the center-of-gravity position of the driver. Thus, it is possible to improve the determination accuracy of the state of the driver while simplifying the configuration of the vehicle.

(12) A driver state determination program according to an embodiment of the present disclosure is a driver state determination program to be used in a driver state determination device. The driver state determination program causes a computer to function as a measurement result acquisition unit configured to acquire a measurement result regarding a change in a center-of-gravity position of a driver of a vehicle, a travel route acquisition unit configured to acquire a travel route of the vehicle, the travel route corresponding to the measurement result, a reference data acquisition unit configured to acquire reference data indicating a criterion for the change in the center-of-gravity position in a target route, the target route being the travel route, and a determination unit configured to perform determination processing of determining a state of the driver, based on the measurement result and the reference data.

With such a configuration, the state of the driver can be accurately determined without performing complicated determination processing, such as determining that the state of the driver is abnormal, for example, when the similarity between the measurement result and the reference data is low, using the measurement result regarding the change in the center-of-gravity position of the driver and the reference data indicating the criterion of the change in the center-of-gravity position in the travel route corresponding to the measurement result. In addition, in the vehicle, it is possible to eliminate the need for an in-vehicle equipment for acquiring other information used for correction of the measurement result, for example, in addition to the in-vehicle equipment for acquiring the measurement result regarding the change in the center-of-gravity position of the driver. Thus, it is possible to improve the determination accuracy of the state of the driver while simplifying the configuration of the vehicle.

Hereinafter, embodiments of the present disclosure will be described with reference to the drawings. In the drawings, the same or corresponding parts are denoted by the same reference signs, and description thereof will not be repeated. At least some of the embodiments described below may be combined as desired.

### Driver State Determination System

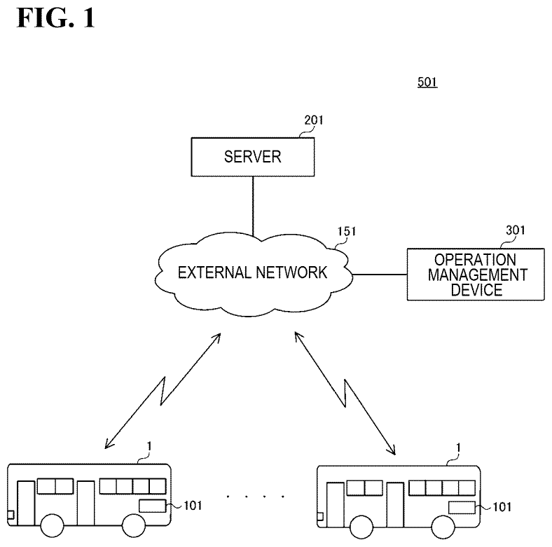

FIG. 1 is a diagram illustrating a configuration of a driver state determination system according to an embodiment of the present disclosure. Referring to FIG. 1, a driver state determination system 501 includes one or more in-vehicle devices 101, a server 201, and an operation management device 301. The in-vehicle device 101 and the server 201 can transmit and receive information via an external network 151 such as the Internet, for example. The server 201 is an example of a driver state determination device. In addition, the server 201 is an example of an external device outside a vehicle 1.

The in-vehicle device 101 is mounted on the vehicle 1. The vehicle 1 is, for example, an automobile having a probe function described later, and is a bus, a truck, a private car, or the like.

The server 201 and the operation management device 301 are used by, for example, a business operator who manages the operation of the vehicle 1 or an individual. The server 201 and the operation management device 301 can transmit and receive information via, for example, the external network 151.

### In-Vehicle Device

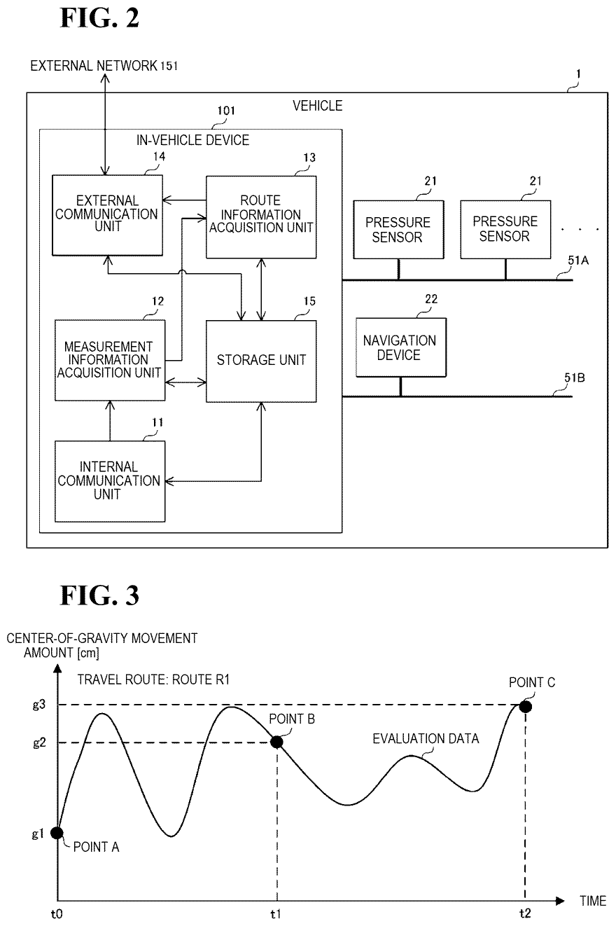

FIG. 2 is a diagram illustrating a configuration of an in-vehicle device according to an embodiment of the present disclosure. Referring to FIG. 2, the in-vehicle device 101 includes an internal communication unit 11, a measurement information acquisition unit 12, a route information acquisition unit 13, an external communication unit 14, and a storage unit 15. Some or all of the internal communication unit 11, the measurement information acquisition unit 12, the route information acquisition unit 13, and the external communication unit 14 are implemented by, for example, processing circuitry including one or more processors. The storage unit 15 is, for example, a nonvolatile memory included in the processing circuitry.

### Measurement Information Acquisition Unit

The measurement information acquisition unit 12 is configured to acquire a center-of-gravity position data corresponding to a measurement result regarding a change in the center-of-gravity position of a driver of the vehicle 1. The center-of-gravity position data is an example of measurement information.

More specifically, for example, a plurality of pressure sensors 21 are provided on a seat surface of a driver's seat of the vehicle 1. The in-vehicle device 101 is connected to the plurality of pressure sensors 21 via a CAN bus 51A that conforms to the standard of controller area network (CAN), for example. The in-vehicle device 101 and each pressure sensor 21 exchange information using a CAN frame that conforms to the standard of CAN.

Each pressure sensor 21 measures a pressure applied to the seat surface of the driver's seat by the driver. Each pressure sensor 21 stores sensor information indicating a measurement value, a measurement time, and an identifier (ID) of the pressure sensor 21 (hereinafter, also referred to as a “sensor ID”) in the CAN frame and transmits to the in-vehicle device 101. Each pressure sensor 21 measures pressure and transmits sensor information, for example, periodically. Here, each pressure sensor 21 measures the pressure at the same measurement time, and transmits the sensor information to the in-vehicle device 101 at the same transmission time, for example.

In the in-vehicle device 101, when the internal communication unit 11 receives the sensor information from each pressure sensor 21, the internal communication unit 11 outputs a plurality of pieces of received sensor information (hereinafter, also referred to as a “sensor information group”) to the measurement information acquisition unit 12.

The storage unit 15 is configured to store a sensor position list indicating a correspondence relationship between the sensor ID and the position of the pressure sensor 21 on the seat surface of the driver's seat.

When the measurement information acquisition unit 12 receives the sensor information group from the internal communication unit 11, the measurement information acquisition unit 12 refers to the sensor position list in the storage unit 15, and thus the position of the pressure sensor 21 corresponding to the sensor ID indicated by the sensor information for each sensor information is specified.

Then, the measurement information acquisition unit 12 calculates the center-of-gravity position of the driver on the seat surface at the corresponding measurement time based on the measurement value indicated by each piece of sensor information and the specified position of each pressure sensor 21. When the measurement information acquisition unit 12 calculates the center-of-gravity position, the measurement information acquisition unit 12 creates the center-of-gravity information indicating the calculated center-of-gravity position and the measurement time, and stores the center-of-gravity information in the storage unit 15. The measurement information acquisition unit 12 creates the center-of-gravity information and stores the center-of-gravity information in the storage unit 15 every time the measurement information acquisition unit 12 receives the sensor information group from the internal communication unit 11. The center-of-gravity position of the driver may be acquired using a camera or an infrared sensor instead of the pressure sensor 21.

Then, when the measurement information acquisition unit 12 creates a predetermined number of pieces of the center-of-gravity information corresponding to a time of, for example, about several minutes to 10 minutes, the measurement information acquisition unit 12 creates center-of-gravity position data using the predetermined number of pieces of the center-of-gravity information stored in the storage unit 15.

Specifically, the measurement information acquisition unit 12 calculates a criterion position that is a criterion for the center-of-gravity position of the driver based on a predetermined number of pieces of the latest center-of-gravity information stored in the storage unit 15. For example, the measurement information acquisition unit 12 calculates an average of the center-of-gravity positions included in the respective pieces of the center-of-gravity information as a criterion position.

When the criterion position is calculated, the measurement information acquisition unit 12 calculates a difference (hereinafter, also referred to as a “center-of-gravity movement amount”) between the calculated criterion position and the center-of-gravity position indicated by the center-of-gravity information for each piece of the center-of-gravity information. Then, the measurement information acquisition unit 12 creates center-of-gravity position data in which the calculated center-of-gravity movement amount and the measurement time indicated by the corresponding center-of-gravity information are integrated, the center-of-gravity position data being time-series center-of-gravity movement information.

When the measurement information acquisition unit 12 creates the center-of-gravity position data, the measurement information acquisition unit 12 outputs the created center-of-gravity position data to the route information acquisition unit 13.

### Route Information Acquisition Unit

The route information acquisition unit 13 is configured to acquire route information indicating the travel route of the vehicle 1 corresponding to the measurement result regarding the change in the center-of-gravity position of the driver of the vehicle 1.

More specifically, the vehicle 1 is provided with, for example, a navigation device 22. The in-vehicle device 101 is connected to the navigation device 22 via a CAN bus 51B, for example. The in-vehicle device 101 and the navigation device 22 exchange information using a CAN frame.

The navigation device 22 creates probe data in which the position of the vehicle 1 during traveling of the vehicle 1 and the time when the vehicle 1 passes the position are integrated, for example. The position of vehicle 1 is, for example, coordinates based on radio waves transmitted from a global positioning system (GPS) satellite. The navigation device 22 creates probe data at each predetermined sampling timing.

When the navigation device 22 creates the probe data, the navigation device 22 transmits the created probe data to the in-vehicle device 101.

In the in-vehicle device 101, when the internal communication unit 11 receives the probe data from the navigation device 22, the internal communication unit 11 stores the received probe data in the storage unit 15.

When the route information acquisition unit 13 receives the center-of-gravity position data from the measurement information acquisition unit 12, the route information acquisition unit 13 acquires a plurality of pieces of probe data (hereinafter, also referred to as a “probe data group”) corresponding to the center-of-gravity position data among the plurality of pieces of probe data stored in the storage unit 15.

Specifically, the probe data group is a plurality of pieces of probe data created in a period from the oldest measurement time to the newest measurement time in the center-of-gravity position data received from the measurement information acquisition unit 12 among the plurality of pieces of probe data stored in the storage unit 15. Then, the route information acquisition unit 13 identifies the travel route of the vehicle 1 that is the trajectory of the positions indicated by the respective probe data belonging to the probe data group.

When the travel route is identified, the route information acquisition unit 13 outputs the center-of-gravity position data, the probe data group, and the route information indicating the travel route to the external communication unit 14.

Hereinafter, the travel route in a case where the vehicle 1 travels from a point A to a point C via a point B is also referred to as a “route R1”, and the travel route in a case where the vehicle 1 travels from the point B to a point E via a point D is also referred to as a “route R2”. The names “route R1” and “route R2” are the same for other vehicles.

The in-vehicle device 101 and the pressure sensors 21, and the in-vehicle device 101 and the navigation device 22 may be configured to perform communication according to a standard such as CAN with flexible data rate (CAN FD), Ethernet (trademark), FlexRay (trademark), Media Oritend System Transport (MOST) (trademark), and local interconnect network (LIN), without being limited to CAN.

### External Communication Unit

The external communication unit 14 configured to transmit the center-of-gravity position data acquired by the measurement information acquisition unit 12 and the probe data acquired by the route information acquisition unit 13 to the server 201.

More specifically, the external communication unit 14 communicates with the server 201 via the external network 151 by performing wireless communication with a device such as a wireless base station device (not illustrated) according to a communication scheme such as WiFi (registered trade mark), LTE (Long Term Evolution) (registered trade mark), or 5G. The external communication unit 14 is not limited to the configuration of communicating with the server 201 via the wireless base station device and the external network 151, and may be configured to communicate with the server 201 via a wired line. The external communication unit 14 may be configured to communicate with the server 201 further via another in-vehicle device.

More specifically, when receiving the center-of-gravity position data, the probe data group, and the route information from the route information acquisition unit 13, the external communication unit 14 creates evaluation data in which the center-of-gravity position data, the probe data group, and the route information are integrated. Specifically, the evaluation data indicates a passing point and a time-series change in the center-of-gravity movement amount when the vehicle 1 travels on the travel route indicated by the route information.

FIG. 3 is a diagram illustrating an example of evaluation data created by the in-vehicle device according to the embodiment of the present disclosure. In FIG. 3, a horizontal axis represents time, and a vertical axis represents center-of-gravity movement amount [cm].

Referring to FIG. 3, the evaluation data indicates, for example, a time-series change in the center-of-gravity movement amount when vehicle 1 travels route R1. The center-of-gravity movement amount at time t0 when the vehicle 1 passes through the point A is “g1”. The center-of-gravity movement amount at time t2 when the vehicle 1 passes through the point B is “g2”. The center-of-gravity shift amount at time t3 when the vehicle 1 passes through the point C is “g3”.

Referring again to FIG. 2, the external communication unit 14 further transmits model information indicating the model of the vehicle 1 and driver identification information (hereinafter also referred to as “driver ID”) for identifying the driver of the vehicle 1 to the server 201.

More specifically, the storage unit 15 stores model information and a driver ID. When the evaluation data is created, the external communication unit 14 transmits determination information including the evaluation data, the model information, and the driver ID to the server 201.

Specifically, for example, the external communication unit 14 creates an IP packet including the determination information and including an IP address of the in-vehicle device 101 and an IP address of the server 201 as a source address and a destination IP address. The external communication unit 14 then transmits the created IP packet to the server 201 via the wireless base station device and the external network 151.

### Server

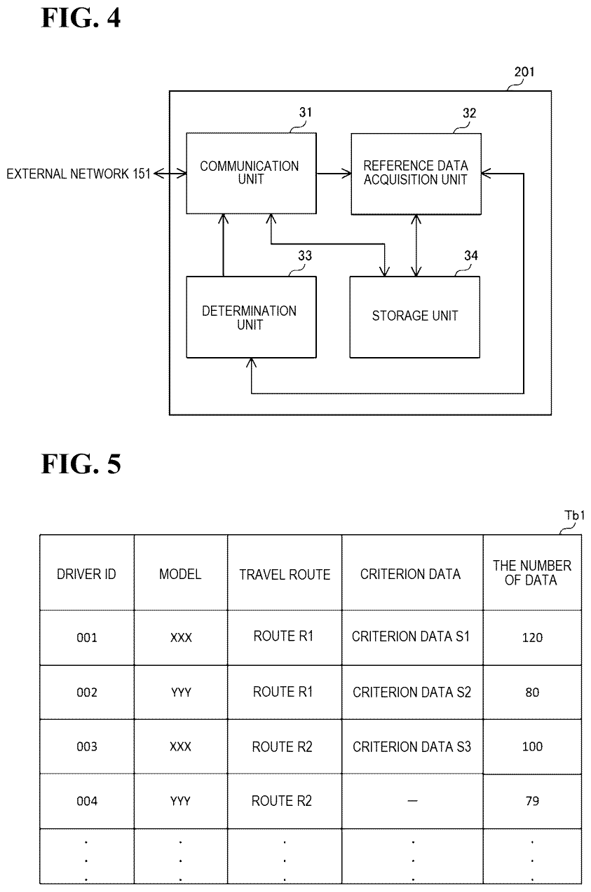

FIG. 4 is a diagram illustrating a configuration of a server according to the embodiment of the present disclosure. Referring to FIG. 4, the server 201 includes a communication unit 31, a reference data acquisition unit 32, a determination unit 33, and a storage unit 34. Some or all of the communication unit 31, the reference data acquisition unit 32, and the determination unit 33 are implemented by, for example, processing circuitry including one or more processors. The storage unit 34 is, for example, a nonvolatile memory included in the processing circuitry. The communication unit 31 is an example of a measurement result acquisition unit and an example of a travel route acquisition unit.

The communication unit 31 acquires a measurement result regarding a change in the center-of-gravity position of the driver of the vehicle 1, specifically, determination information. The communication unit 31 acquires the travel route of the vehicle 1 corresponding to the acquired determination information.

More specifically, when the communication unit 31 receives the IP packet including the determination information from the in-vehicle device 101 via the wireless base station device and the external network 151, the communication unit 31 adds a data ID to the determination information and stores the determination information in the storage unit 34, and outputs notification of acquisition indicating that the determination information has been acquired and the data ID to the reference data acquisition unit 32.

The reference data acquisition unit 32 acquires reference data indicating a criterion for a change in the center-of-gravity position of the driver of the vehicle 1 in the travel route (hereinafter also referred to as a “target route”) acquired by the communication unit 31. For example, the reference data indicates a criterion for a change in the center-of-gravity position of the driver of the vehicle 1 in a case where a vehicle of the same model as the vehicle 1 travels on the target route.

More specifically, the reference data indicates a criterion for a change in the center-of-gravity position when a vehicle of the same model as the model indicated by the model information included in the determination information acquired by the communication unit 31 travels on the target route indicated by the evaluation data included in the determination information.

FIG. 5 is a diagram illustrating an example of a correspondence table stored in the server according to the embodiment of the present disclosure.

Referring to FIG. 4 and FIG. 5, for example, the reference data acquisition unit 32 acquires a correspondence table Tb1 indicating a correspondence relationship among a driver ID, a model, a travel route, and criterion data indicating a criterion for a change of a center-of-gravity position of a driver of the vehicle 1 in the travel route. Then, the reference data acquisition unit 32 acquires criterion data based on the acquired correspondence table Tb1. The correspondence table Tb1 is an example of correspondence information.

More specifically, for example, the storage unit 34 stores the correspondence table Tb1. In the correspondence table Tb1, the criterion data corresponding to the driver ID “001”, the model “XXX”, and the travel route “route R1” is “criterion data S1”. The criterion data corresponding to the driver ID “002”, the model “YYY”, and the travel route “route R1” is “criterion data S2”. The criterion data corresponding to the driver ID “003”, the model “XXX”, and the travel route “route R2” is “criterion data S3”. The criterion data corresponding to the driver ID “004”, the model “YYY”, and the travel route “route R2” is “not applicable”. The “number of data” illustrated in FIG. 5 will be described later.

For example, the communication unit 31 acquires the determination information as a measurement result (hereinafter also referred to as a “target measurement result”) regarding a change in the center-of-gravity position of the driver of the vehicle 1 in the target route. The criterion data may be created based on the target measurement result acquired by the communication unit 31. Here, the driver of the vehicle 1 is a driver that is a target of determination processing described later.

Here, for example, the reference data acquisition unit 32 performs data creation processing of creating criterion data based on the target measurement result acquired by the communication unit 31.

More specifically, the reference data acquisition unit 32 creates criterion data based on the evaluation data included in the determination information acquired by the communication unit 31. That is, the evaluation data may be source data of the criterion data.

Specifically, the correspondence table Tb1 indicates a correspondence relationship between the criterion data and the number of source data accumulated in the storage unit 34 (hereinafter also referred to as “the number of data K”), in addition to a correspondence relationship between the driver ID, the model, the travel route, and the criterion data.

In the correspondence table Tb1 illustrated in FIG. 5, the number of data K corresponding to the driver ID “1”, the model “XXX”, and the travel route “route R1” is “120”. The number of data K corresponding to the driver ID “002”, the model “YYY”, and the travel route “route R1” is “80”. The number of data K corresponding to the driver ID “003”, the model “XXX”, and the travel route “route R2” is “100”. The number of data K corresponding to the driver ID “004”, the model “YYY”, and the travel route “route R2” is “79”.

When the reference data acquisition unit 32 receives acquisition notification from the communication unit 31, the reference data acquisition unit 32 acquires, from the storage unit 34, the correspondence table Tb1 and determination information (hereinafter also referred to as “determination information S”) to which the data ID indicated by the acquisition notification is added. Then, the reference data acquisition unit 32 identifies the driver ID, the model indicated by the model information, and the target route indicated by the evaluation data included in the determination information S. The reference data acquisition unit 32 refers to the correspondence table Tb1 to determine whether the number of data K corresponding to the identified driver ID, model, and target route is equal to or greater than a predetermined threshold value Th1.

### When the Number of Data is Less than the Threshold

When the number of data K is less than the threshold Th1, the reference data acquisition unit 32 determines whether or not the evaluation data included in the determination information S satisfies the predetermined condition.

For example, when the determination information S acquired by the communication unit 31 satisfies the predetermined condition, the reference data acquisition unit 32 does not use the determination information S as the source data of the criterion data.

More specifically, for example, when the reference data acquisition unit 32 determines that the number of data K is less than the threshold Th1, the reference data acquisition unit 32 calculates the change amount of the center-of-gravity movement amount per predetermined unit time based on the evaluation data included in the determination information S.

When the calculated change amount is equal to or greater than a predetermined threshold value Th2, the reference data acquisition unit 32 determines that the change amount is a change amount caused by an abnormal behavior of the driver (hereinafter, also referred to as an “abnormality change amount”). Here, the abnormal behavior is, for example, sudden braking, sudden steering, or the like.

When the reference data acquisition unit 32 determines that the calculated change amount is the abnormality change amount, the reference data acquisition unit 32 determines not to use the evaluation data included in the determination information S as the source data of the criterion data. Then, the reference data acquisition unit 32 discards the determination information S from the storage unit 34.

On the other hand, when the calculated change amount is less than the threshold Th2, the reference data acquisition unit 32 determines that the change amount is not an abnormality change amount. Then, the reference data acquisition unit 32 determines to use the evaluation data included in the determination information S as the source data of the criterion data. Hereinafter, the evaluation data used as the source data of the criterion data is also referred to as “evaluation data Da”.

### Registration of Evaluation Data

The storage unit 34 stores a management table Tb2 indicating source data of the criterion data. Specifically, the management table Tb2 indicates a correspondence relationship among the driver ID, the model, the travel route, and the evaluation data.

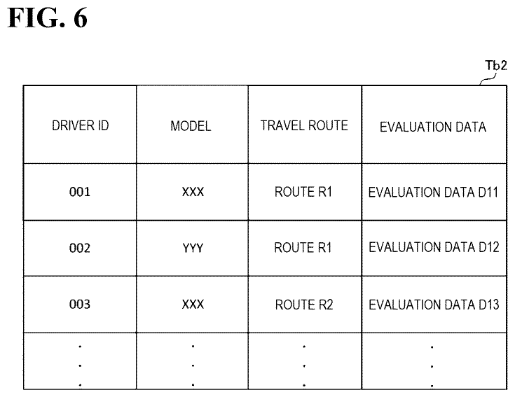

FIG. 6 is a diagram illustrating an example of a management table stored in the server according to the embodiment of the present disclosure.

Referring to FIG. 6, in the management table Tb2, for example, the evaluation data “evaluation data D11” corresponding to the driver ID “001”, the model “XXX”, and the travel route “route R1”, the evaluation data “evaluation data D12” corresponding to the driver ID “002”, the model “YYY”, and the travel route “route R1”, and the evaluation data “evaluation data D13” corresponding to the driver ID “003”, the model “XXX”, and the travel route “route R2” are registered as the source data of the criterion data.

Referring again to FIG. 4, when the reference data acquisition unit 32 determines to use the evaluation data Da included in the determination information S as the source data of the criterion data, the reference data acquisition unit 32 reads the management table Tb2 in the storage unit 34. Then, the reference data acquisition unit 32 registers the evaluation data Da in the management table Tb2 in association with the driver ID included in the determination information S, the model indicated by the model information, and the target route indicated by the evaluation data Da.

### Update of the Number of Data

When the reference data acquisition unit 32 determines to use the evaluation data Da included in the determination information S as the source data of the criterion data, the reference data acquisition unit 32 updates the number of data K in the correspondence table Tb1 illustrated in FIG. 5. Hereinafter, the criterion data corresponding to the evaluation data Da is also referred to as “criterion data St”, and the source data of the criterion data St is also referred to as “source data Dn”. Each piece of the source data Dn is, for example, data indicating the same probe data group and route information.

Specifically, the reference data acquisition unit 32 adds one to the number of data K corresponding to the driver ID included in the determination information S, the model of the model information, and the target route indicated by the evaluation data Da in the correspondence table Tb1. The reference data acquisition unit 32 then determines whether the number of data K after adding is equal to or greater than the threshold Th1.

When the number of data K after adding is less than the threshold Th1, the reference data acquisition unit 32 does not perform the data creation processing.

On the other hand, when the number of data K after adding is equal to or greater than the threshold Th1, the reference data acquisition unit 32 performs data creation processing.

More specifically, for example, when the number of data K after adding is equal to or greater than the threshold Th1, the reference data acquisition unit 32 acquires, from the storage unit 34, other evaluation data other than the evaluation data Da among the source data Dn of the criterion data St. Then, the reference data acquisition unit 32 creates criterion data St based on the evaluation data Da and the other evaluation data.

Specifically, the reference data acquisition unit 32 calculates an average value of the center-of-gravity movement amount in each measurement time based on the center-of-gravity position data indicated by each piece of the source data Dn, for example. Then, the reference data acquisition unit 32 creates the averaged data indicating a time-series change in the average value of the center-of-gravity movement amount, in association with the calculated average value and the measurement time.

The reference data acquisition unit 32 creates the criterion data St in which a probe data group and route information common to each piece of the source data Dn are associated with the created averaged data. The reference data acquisition unit 32 may select any one of the source data Dn as the criterion data St according to some criterion, not limited to the average.

When the criterion data St is created, the reference data acquisition unit 32 stores the created criterion data St in the storage unit 34 and registers the created criterion data St in the correspondence table Tb1 in the storage unit 34.

In the example illustrated in FIG. 5, when the number of data K corresponding to the driver ID “004”, the model “YYY” and the travel route “route R2” reaches the threshold Th1 or more and criterion data S4 is created, the reference data acquisition unit 32 stores the criterion data S4 in the storage unit 34 and registers the criterion data S4 in the correspondence table Tb1.

### When the Number of Data is Equal to or Greater than the Threshold

When the number of data K is equal to or greater than the threshold Th1, the reference data acquisition unit 32 refers to the correspondence table Tb1 to specify criterion data corresponding to the target route indicated by the evaluation information included in the determination information S.

When the criterion data is specified, the reference data acquisition unit 32 acquires the specified criterion data from the storage unit 34. Then, the reference data acquisition unit 32 outputs, to the determination unit 33, determination request information including the evaluation data included in the determination information S and the criterion data acquired from the storage unit 34.

The correspondence table Tb1 illustrated in FIG. 5 may be configured not to include the item of “driver ID”. The correspondence table Tb1 may be configured to include the item of “driver ID”, and may be configured to include data such as criterion data by inputting Null as the data of the driver ID. In this case, for example, when a plurality of pieces of criterion data corresponding to the target route indicated by the evaluation data are registered in the correspondence table Tb1, the reference data acquisition unit 32 acquires data obtained by weighting and adding each piece of criterion data as the reference data.

### Determination Processing

The determination unit 33 performs determination processing of determining the state of the driver of the vehicle 1 based on the determination information S acquired by the communication unit 31 and the criterion data acquired by the reference data acquisition unit 32.

More specifically, for example, the determination unit 33 performs a processing of determining the abnormality of the driver of the vehicle 1 as the determination processing. Here, the abnormality of the driver is an abnormality that may lead to an accident of the vehicle 1, such as fatigue and drowsiness.

Specifically, for example, when the determination unit 33 receives the determination request information from the reference data acquisition unit 32, the determination unit 33 performs a comparison processing of comparing the evaluation data included in the determination information S with the criterion data included in the determination request information. A determination processing performed based on a comparison result between the cross-correlation coefficient and the threshold Th3, which will be described below, is also referred to as a first determination processing.

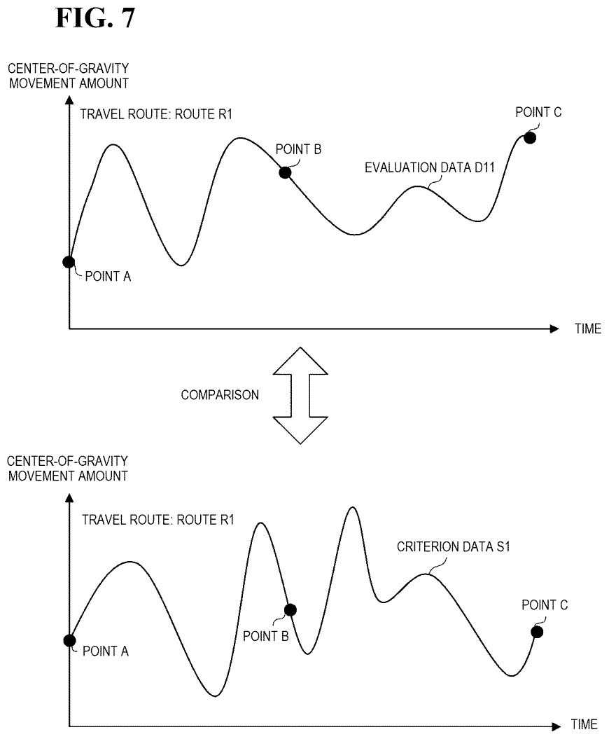

FIG. 7 is a diagram for explaining an example of a comparison processing by the server according to the embodiment of the present disclosure;

The upper part of FIG. 7 illustrates the evaluation data D11 that is an example of the evaluation data used in the comparison processing. The lower part of FIG. 7 illustrates an example of criterion data S1 that is an example of criterion data used in the comparison processing.

Referring to FIGS. 4 and 7, for example, the determination unit 33 compares the evaluation data D11 with the criterion data S1, and calculates a cross-correlation coefficient between the evaluation data D11 and the criterion data S1 as a comparison result. The cross-correlation coefficient indicates similarity between the evaluation data D11 and the criterion data S1.

The cross-correlation coefficient is calculated as a value of 0 to 1, for example. For example, in a case where the cross-correlation coefficient is close to 1, the evaluation data D11 is similar to the criterion data S1. On the other hand, in a case where the cross-correlation coefficient is close to 0, the evaluation data D11 is not similar to the criterion data S1.

When the cross-correlation coefficient is calculated, the determination unit 33 determines whether the calculated cross-correlation coefficient is less than a predetermined threshold value Th3. The threshold Th3 is, for example, a value greater than 0 and less than 1.

When the calculated cross-correlation coefficient is less than the threshold Th3, the determination unit 33 determines that the state of the driver is abnormal. Then, the determination unit 33 outputs abnormality notification indicating that the state of the driver is abnormal to the communication unit 31.

Referring again to FIGS. 1, 2, and 4, when the communication unit 31 receives the abnormality notification from the determination unit 33, the communication unit 31 transmits the abnormality notification to the operation management device 301 via the external network 151.

When the operation management device 301 receives the abnormality notification from the server 201 via the external network 151, the operation management device 301 displays the received abnormality notification on its own monitor or the like.

Further, when the communication unit 31 receives the abnormality notification from the determination unit 33, the communication unit 31 transmits the IP packet including the abnormality notification to the navigation device 22 of the vehicle 1 via the external network 151 and the wireless base station device.

When the navigation device 22 receives the abnormality notification from the server 201 via the external network 151 and the wireless base station device, the navigation device 22 displays the received abnormality notification on its own monitor or the like.

On the other hand, when the calculated cross-correlation coefficient is equal to or greater than the threshold Th3, the determination unit 33 determines that the state of the driver is normal.

The determination unit 33 is not limited to a configuration in which the determination processing is performed based on the comparison result between the cross-correlation coefficient and the threshold Th3, and may be configured to perform a determination processing (hereinafter, also referred to as a second determination processing) using a learning model L that outputs a determination result regarding the state of the driver of the vehicle 1.

More specifically, for example, the determination unit 33 generates the learning model L by supervised learning. Specifically, for example, the determination unit 33 acquires in advance a training data set including the evaluation data when the state of the driver is normal and the evaluation data when the state of the driver is abnormal, and generates the learning model L using the training data set. Then, the determination unit 33 stores the generated learning model L in the storage unit 34.

When the determination unit 33 receives the determination request information from the reference data acquisition unit 32, the determination unit 33 acquires the learning model L from the storage unit 34. Then, the determination unit 33 inputs the evaluation data included in the determination information S and the criterion data included in the determination request information to the learning model L, and acquires the determination result output from the learning model L.

When the determination result indicating that the state of the driver is abnormal is output from the learning model L, the determination unit 33 transmits abnormality occurrence information to the operation management device 301 and the navigation device 22. On the other hand, when the determination result indicating that the state of the driver is normal is output from the learning model L, the determination unit 33 outputs data normality information to the reference data acquisition unit 32.

The determination unit 33 may perform a determination processing (hereinafter also referred to as a third determination processing) using the number of points at which the center-of-gravity movement amount equal to or greater than a predetermined threshold value Th4 is measured.

Specifically, for example, in the data creation processing, the reference data acquisition unit 32 counts the number of points (hereinafter, also referred to as an “abnormality point”) at which the center-of-gravity movement amount equal to or greater than the threshold Th4 is measured for each piece of the evaluation data. Then, the reference data acquisition unit 32 stores the average value of the number of abnormality points in a plurality of pieces of the evaluation data as an abnormality point average value in the correspondence table Tb1 in association with the corresponding criterion data.

In the third determination processing, the determination unit 33 counts the number of abnormality points in the evaluation data included in the determination information S. The determination unit 33 calculates a difference between the counted number and the number of the abnormality points counted in the criterion data (or the average value of the number of the abnormality points counted in the source data of the criterion data), and determines whether the calculated difference is equal to or greater than a predetermined threshold value Th5.

When the calculated difference is equal to or greater than the threshold Th5, the determination unit 33 determines that the state of the driver is abnormal. On the other hand, when the calculated difference is less than the threshold Th5, the determination unit 33 determines that the state of the driver is normal.

### Update of Criterion Data

Referring again to FIG. 4, for example, when the determination unit 33 determines that the state of the driver is normal, the determination unit 33 outputs a normality notification indicating that the state is normal, and the data normality information including the evaluation data D11 and the criterion data S1 used in the determination processing to the reference data acquisition unit 32.

When the reference data acquisition unit 32 receives the data normal information from the determination unit 33, the reference data acquisition unit 32 determines to use the evaluation data D11 included in the data normality information as source data of new criterion data.

More specifically, for example, when the reference data acquisition unit 32 receives the data normality information from the determination unit 33, the reference data acquisition unit 32 performs a table update processing of registering the evaluation data included in the data normality information in the management table Tb2.

Specifically, when receiving the data normality information from the determination unit 33, the reference data acquisition unit 32 reads the correspondence table Tb1 in the storage unit 34. Then, the reference data acquisition unit 32 refers to the correspondence table Tb1 to identify the driver ID, the model, and the travel route corresponding to the criterion data S1 included in the data normality information.

Then, the reference data acquisition unit 32 performs, as the table update processing, the processing of registering the evaluation data included in the data normality information, and the identified driver ID, model, and travel route in the management table Tb2.

Hereinafter, evaluation data D50 will be described as an example of the evaluation data included in the data normality information. In addition, the driver ID “050”, the model “ZZZ”, and the travel route “route R2” will be described as examples of the driver ID, the model, and the travel route corresponding to the criterion data included in the data normality information, respectively.

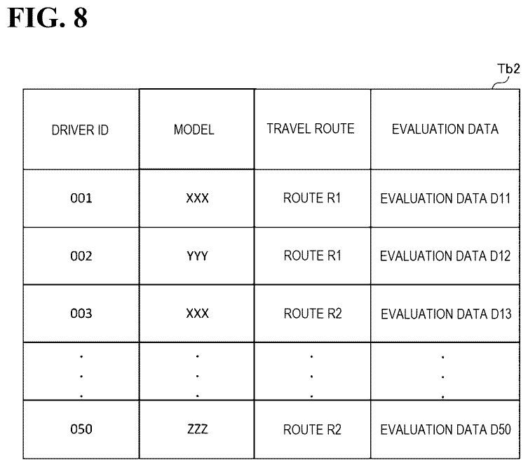

FIG. 8 is a diagram illustrating an example of a management table after a table update processing by the server according to the embodiment of the present disclosure.

Referring to FIG. 8, in the updated management table Tb2, the evaluation data “evaluation data D50” corresponding to the driver ID “050”, the model “ZZZ”, and the travel route “route R2” is newly registered as the source data of the criterion data.

Referring again to FIGS. 4 and 5, the reference data acquisition unit 32 updates the criterion data after the table update processing.

More specifically, for example, the criterion data is created based on the target measurement result and the measurement result in the case where the determination unit 33 determines that the abnormality does not occur, among a plurality of measurement results acquired by the communication unit 31.

In detail, when the reference data acquisition unit 32 receives the data normality information from the determination unit 33, the reference data acquisition unit 32 identifies the criterion data S1 included in the data normality information, and acquires, from the storage unit 34, other evaluation data other than the evaluation data D11 among the source data of the criterion data S1 stored in the storage unit 34.

Then, the reference data acquisition unit 32 creates new criterion data by the same method as the data creation processing described above, stores the created new criterion data in the storage unit 34, and registers the created criterion data St in the correspondence table Tb1 in the storage unit 34.

### Flow of Operation

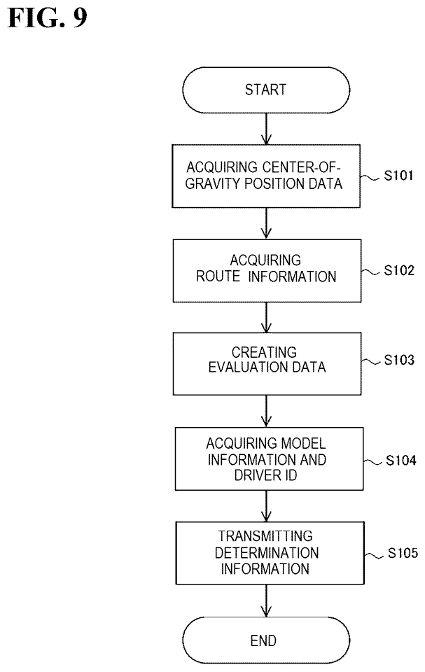

FIG. 9 is a flowchart illustrating an operation procedure when the in-vehicle device according to the embodiment of the present disclosure performs a processing of transmitting the determination information S.

Referring to FIG. 9, first, the in-vehicle device 101 acquires the center-of-gravity position data corresponding to a measurement result regarding a change in the center-of-gravity position of the driver of the vehicle 1. For example, as described above, the in-vehicle device 101 creates the center-of-gravity position data using a predetermined number of pieces of the latest center-of-gravity information stored in the storage unit 15 (step S101).

Next, the in-vehicle device 101 acquires route information indicating the travel route of the vehicle 1 corresponding to the created center-of-gravity position data. For example, as described above, the in-vehicle device 101 acquires probe data group created in a period from the oldest measurement time to the newest measurement time in the center-of-gravity position data among the plurality of pieces of probe data stored in the storage unit 15. Then, the in-vehicle device 101 identifies a travel route indicating a trajectory of positions indicated by the respective probe data belonging to the acquired probe data group (step S102).

Next, the in-vehicle device 101 creates evaluation data in which the center-of-gravity position data, the probe data group, and the route information indicating the travel route are associated with each other (step S103).

Next, the in-vehicle device 101 acquires model information indicating the model of the vehicle 1 and the driver ID from the storage unit 15 (step S104).

Next, the in-vehicle device 101 transmits the evaluation data and the determination information S including the model information and the driver ID to the server 201 (step S105).

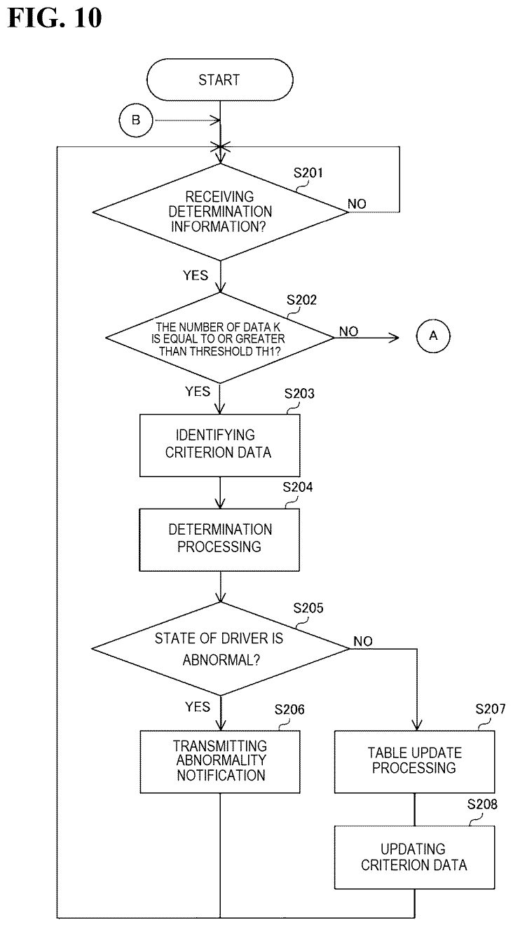

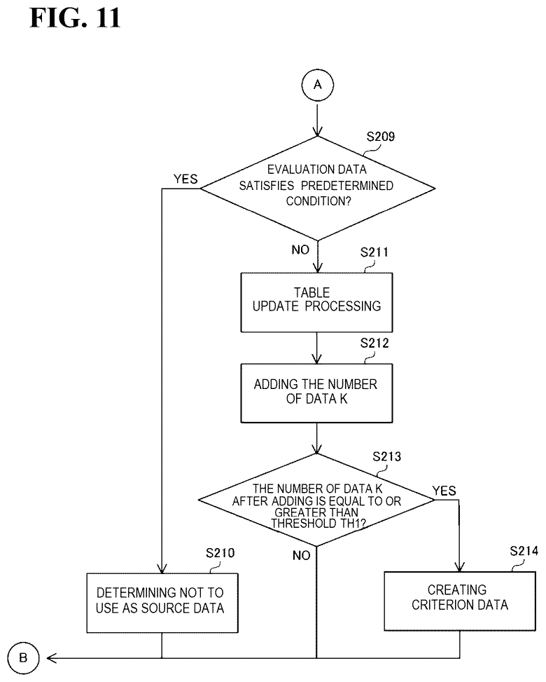

FIG. 10 and FIG. 11 are flowcharts illustrating the operation procedure when the server according to the embodiment of the present disclosure performs the determination processing.

Referring to FIG. 10 and FIG. 11, first, the server 201 waits for determination information S from the in-vehicle device 101 (NO in step S201), and when the server 201 receives the determination information S from the in-vehicle device 101 (YES in step S201), the server 201 refers to the correspondence table Tb1 in the storage unit 34 to determine whether or not the number of data K corresponding to the driver ID included in the determination information S, the model indicated by the model information, and the target route indicated by the evaluation information is equal to or greater than the threshold Th1 (step S202).

When the number of data K is equal to or greater than the threshold Th1 (YES in step S202), the server 201 refers to the correspondence table Tb1 in the storage unit 34 to specify the criterion data corresponding to the evaluation data included in the determination information S (step S203).

Next, the server 201 performs the determination processing of determining the state of the driver of the vehicle 1 based on the evaluation data included in the determination information S and the specified criterion data (step S204).

Next, when the server 201 determines that the state of the driver of the vehicle 1 is abnormal in the determination processing (YES in step S205), the server 201 transmits the abnormality notification indicating that the state is abnormal to the operation management device 301 and the navigation device 22 provided in the vehicle 1 (step S206), and waits for new determination information S from the in-vehicle device 101 (step S201).

On the other hand, when the server 201 determines that the state of the driver of the vehicle 1 is normal in the determination processing (NO in step S205), the server 201 determines to use the evaluation data used in the determination processing as the source data of the criterion data. Then, the server 201 performs the table update processing of registering the evaluation data in the management table Tb2 in the storage unit 34 (step S207).

Next, the server 201 updates the criterion data using the evaluation data determined to be used as the source data of the criterion data. For example, as described above, the server 201 creates new criterion data based on the evaluation data and other evaluation data stored in the storage unit 34 (step S208), and waits for new determination information S from the in-vehicle device 101 (step S201).

When the number of data K is less than the threshold Th1 (NO in step S202), the server 201 determines whether or not the evaluation data included in the determination information S received from the in-vehicle device 101 satisfies the predetermined condition. Here, the server 201 determines whether or not the evaluation data D11 included in the determination information S satisfies the predetermined condition (step S209).

Then, when the evaluation data D11 satisfies the predetermined condition (YES in step S209), the server 201 determines not to use the evaluation data D11 as the source data of the criterion data. For example, as described above, the server 201 discards the determination information S including the evaluation data D11 from the storage unit 34 (step S210), and waits for new determination information S from the in-vehicle device 101 (step S201).

On the other hand, when the evaluation data D11 does not satisfy the predetermined condition (NO in step S209), the server 201 determines to use the evaluation data D11 as the source data of the criterion data. Then, the server 201 performs the table update processing of registering the evaluation data D11 in the management table Tb2 in the storage unit 34 (step S211).

Next, the server 201 adds one to the number of data K corresponding to the driver ID included in the determination information S, the model indicated by the model information, and the target route indicated by the evaluation data D11 in the correspondence table Tb1 in the storage unit 34 (step S212).

Next, when the number of data K after adding is equal to or greater than the threshold Th1 (YES in step S213), the server 201 creates the criterion data based on the evaluation data D11 and other evaluation data other than the evaluation information D11 stored in the storage unit 34 (step S214), and waits for new determination information S from the in-vehicle device 101 (step S201).

On the other hand, when the number of data K after adding is less than the threshold Th1 (NO in step S213), the server 201 waits for new determination information S from the in-vehicle device 101 (step S201 shown in FIG. 10).

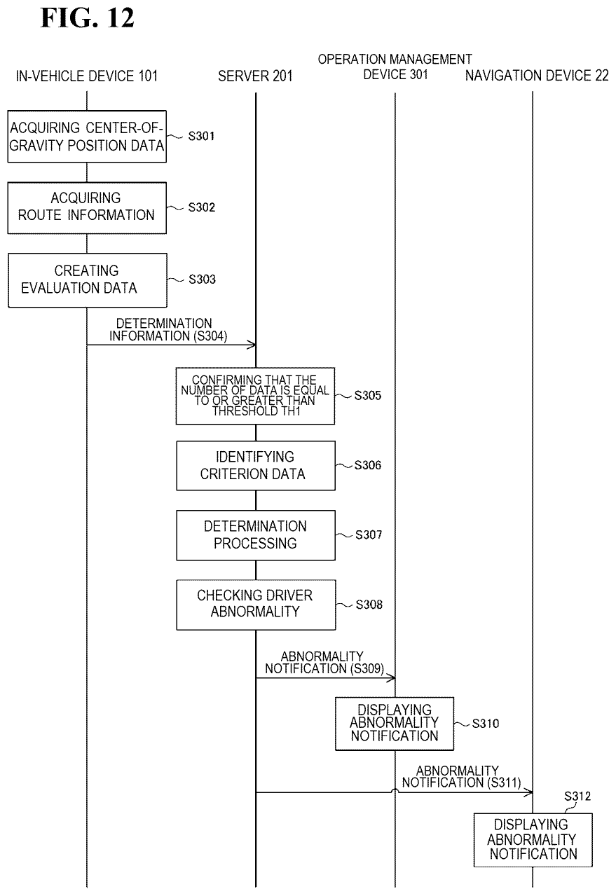

FIG. 12 is a diagram illustrating an example of a sequence of processing of an in-vehicle device, a server, an operation management device, and a navigation device in the driver state determination system according to the embodiment of the present disclosure.

Referring to FIG. 12, first, the in-vehicle device 101 acquires center-of-gravity position data corresponding to a measurement result regarding a change in the center-of-gravity position of the driver of the vehicle 1. For example, as described above, the in-vehicle device 101 creates the center-of-gravity position data using a predetermined number of pieces of the latest center-of-gravity information stored in the storage unit 15 (step S301).

Next, the in-vehicle device 101 acquires route information indicating the travel route of the vehicle 1 corresponding to the created center-of-gravity position data. For example, as described above, the in-vehicle device 101 acquires the probe data group created in a period from the oldest measurement time to the newest measurement time in the center-of-gravity position data among the plurality of probe data stored in the storage unit 15. Then, the in-vehicle device 101 identifies a travel route indicating a trajectory of positions indicated by the respective probe data belonging to the acquired probe data group (step S302).

Next, the in-vehicle device 101 creates evaluation data in which the center-of-gravity position data, the probe data group, and the route information indicating the identified travel route are associated with each other (step S303).

Next, the in-vehicle device 101 transmits the determination information S including the created evaluation data, the model information indicating the model of the vehicle 1, and the driver ID to the server 201 (step S304).

Next, the server 201 refers to the correspondence table Tb1 stored in the storage unit 34 to determine whether the number of data K corresponding to the driver ID included in the determination information S, the model indicated by the model information, and the target route indicated by the evaluation data is equal to or greater than the threshold Th1. Here, it is assumed that the server 201 determines that the number of data K is equal to or greater than the threshold Th1 (step S305).

Next, the server 201 refers to the correspondence table Tb1 in the storage unit 34 to specify criterion data corresponding to the evaluation data included in the determination information S (step S306).

Next, the server 201 performs determination processing of determining the abnormality of the driver of the vehicle 1 based on the evaluation data included in the determination information S and the specified criterion data (step S307).

Next, the server 201 determines that the state of the driver of the vehicle 1 is abnormal in the determination processing (step S308).

Next, the server 201 transmits the abnormality notification indicating that the state of the driver of the vehicle 1 is abnormal to the operation management device 301 (step S309).

Next, when the operation management device 301 receives the abnormality notification from the server 201, the operation management device 301 displays the received abnormality notification on its own monitor or the like (step S310).

Next, the server 201 transmits the abnormality notification to the navigation device 22 provided in the vehicle 1 (step S311).

Next, when the navigation device 22 receives the abnormality notification from the server 201, the navigation device 22 displays the received abnormality notification on its own monitor or the like (step S312). The processing of step S311 and step S312 may be executed by changing the order of the processing of step S309 and step S310, or may be executed in parallel.

### Modification

The external communication unit 14 in the in-vehicle device 101 illustrated in FIG. 2 may be configured to transmit the determination information not including the driver ID, that is, the determination information including the evaluation data and the model information to the server 201. Alternatively, the external communication unit 14 may be configured to transmit the determination information including the driver ID and not to include the driver ID in the correspondence table Tb1. The correspondence table Tb1 may be configured to include the driver ID, and may be configured to include data such as criterion data by inputting Null as the data of the driver ID. In the server 201, when the criterion is updated, the reference data acquisition unit 32 may perform data-adjustment processing of associating the passing time at the predetermined point indicated by the criterion data before the update with the passing time at the predetermined point indicated by the evaluation data D50.

More specifically, for example, the speed of the vehicle during driving may differ for each driver. In this case, the time at which the vehicle passes through each point in the travel route differs for each driver.

Thus, in the modification, the reference data acquisition unit 32 performs a data adjustment processing when the criterion data is updated.

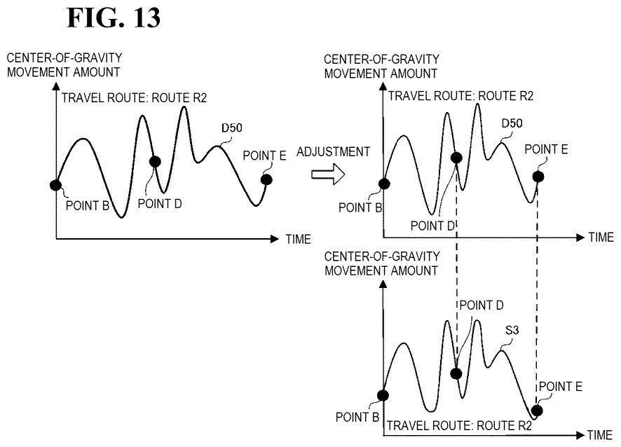

FIG. 13 is a diagram for explaining the data adjustment processing by the modification of the server according to the embodiment of the present disclosure. In FIG. 13, the graph on the left side of the upper part of the diagram illustrates the evaluation data D50 before the data adjustment processing. The graph on the right side of the upper part of the diagram illustrates the evaluation data D50 after the data adjustment processing. The graph in the lower part of the diagram illustrates the criterion data S3 corresponding to the evaluation data D50.

The model corresponding to the criterion data S3 is the same as the model corresponding to the evaluation data D50. The travel route indicated by the criterion data S3 is the same “route R2” as the travel route indicated by the evaluation data D50.

Referring to FIG. 13, the reference data acquisition unit 32 performs a processing of reducing or enlarging the horizontal axis of the graph indicating the evaluation data D50 so that the passing times at the point D and the point E indicated by the evaluation data D50 become the same as the passing times at the point D and the point E indicated by the criterion data S3, respectively, and in particular, corrects each measurement time in the evaluation data D50.

In the example illustrated in FIG. 13, in the evaluation data D50 before the data adjustment processing, the passing times at the point D and the point E are later than the passing times at the point D and the point E indicated by the criterion data S3, respectively. Thus, the reference data acquisition unit 32 performs a processing of reducing the horizontal axis in the graph on the left side of the upper part of FIG. 13 in the data adjustment processing.

The reference data acquisition unit 32 stores the evaluation data D50 after the data adjustment processing in the storage unit 34, and updates the criterion data by the same method as the data creation processing described above.

The driver state determination system 501 according to the embodiment of the present disclosure is configured to create the reference data based on the measurement result regarding the change in the center-of-gravity position of the same driver as the driver of the vehicle 1 in the same travel route as the target route, but the present disclosure is not limited thereto. The driver state determination system 501 may be configured to create the reference data based on the measurement result regarding the change in the center-of-gravity position of a driver different from the driver of the vehicle 1 in the same travel route as the target route.

The driver state determination system 501 according to the embodiment of the present disclosure is configured to create the reference data based on the measurement result regarding the change in the center-of-gravity position of the driver of the vehicle 1 in the target route, but the present disclosure is not limited thereto. The driver state determination system 501 may be configured to create the reference data based on the measurement result regarding the change in the center-of-gravity position of the driver of the vehicle 1 in a travel route other than the target route.

The server 201 according to the embodiment of the present disclosure is configured to update the reference data based on the target measurement result acquired by the communication unit 31 and the measurement result when the determination unit 33 determines that the abnormality does not occur, but the present disclosure is not limited thereto. The server 201 may be configured not to update the reference data after creating once.

In the server 201 according to the embodiment of the present disclosure, the reference data acquisition unit 32 is configured not to use the evaluation data as the source data of the criterion data when the evaluation data included in the determination information S acquired by the communication unit 31 satisfies the predetermined condition, but the present disclosure is not limited thereto. The reference data acquisition unit 32 may be configured to use the evaluation data as the source data of the criterion data regardless of whether the evaluation data included in the determination information S satisfies the predetermined condition.

In the server 201 according to the embodiment of the present disclosure, the reference data acquisition unit 32 is configured to acquire, as the reference data, the criterion data indicating the criterion for the change in the center-of-gravity position of the driver when the vehicle of the same model as the vehicle 1 travels on the target route, but the present disclosure is not limited thereto. The reference data acquisition unit 32 may be configured to acquire, as the reference data, the criterion data indicating a criterion for the change in the center-of-gravity position of the driver when a vehicle that is a model different from the model of the vehicle 1 travels on the target route.

In the driver state determination system 501 according to the embodiment of the present disclosure, the server 201 is configured to include the storage unit 34 configured to store the correspondence table Tb1, but the present disclosure is not limited thereto. A device other than the server 201 may be configured to store the correspondence table Tb1, and the server 201 may be configured to receive notification regarding the criterion data corresponding to the target route indicated by the evaluation data from another device.

In the driver state determination system 501 according to the embodiment of the present disclosure, the server 201 is configured to create the reference data, but the present disclosure is not limited thereto. The server 201 may be configured to acquire the reference data created by a device other than the server 201.

In addition, some or all of the functions of the server 201 according to the embodiment of the present disclosure may be provided by cloud computing. That is, the server 201 according to the embodiment of the present disclosure may be a cloud server including a plurality of servers.

The above embodiments are to be considered in all respects as illustrative and not restrictive. The scope of the present invention is defined by the appended claims rather than by the foregoing description, and is intended to include any modifications within the scope and meaning equivalent to the scope of the claims.

Each processing (each function) of the above-described embodiment is implemented by processing circuitry including one or more processors. The processing circuitry may be configured by one or more memories, an integrated circuit in which various analog circuits and various digital circuits are combined, and the like, in addition to one or more processors. The one or more memories store a program (instructions) that causes the one or more processors to execute each processing. The one or more processors may execute each processing according to a program read from the one or more memories, or may execute each processing according to a logic circuit designed to execute each processing in advance. The processor may be various processors suitable for control of a computer, such as a central processing unit (CPU), a graphics processing unit (GPU), a digital signal processor (DSP), a field programmable gate array (FPGA), and an application specific integrated circuit (ASIC). A plurality of physically separated processors may execute each processing in cooperation with each other. For example, processors mounted on a plurality of physically separated computers may execute each processing in cooperation with each other via a network such as a local area network (LAN), a wide area network (WAN), and the Internet. The program may be installed in the memory from an external server device or the like via a network, or may be distributed in a state of being stored in a recording medium such as a compact disc read only memory (CD-ROM), a digital versatile disk read only memory (DVD-ROM), or a semiconductor memory, and may be installed in the memory from the recording medium.

The above description includes the following features.

### Appendix 1

A driver state determination device including:
a measurement result acquisition unit configured to acquire a measurement result regarding a change in a center-of-gravity position of a driver of a vehicle;
a travel route acquisition unit configured to acquire a travel route of the vehicle, the travel route corresponding to the measurement result;
a reference data acquisition unit configured to acquire reference data indicating a criterion for the change in the center-of-gravity position in a target route, the target route being the travel route; and
a determination unit configured to perform determination processing of determining a state of the driver, based on the measurement result and the reference data, wherein
the measurement result acquisition unit is configured to acquire a plurality of pieces of measurement data each corresponding to a respective one of a plurality of the measurement results,
the reference data acquisition unit is configured to perform data creation processing of creating the reference data, based on the plurality of pieces of measurement data,
the driver state determination device further includes
a storage unit configured to store the plurality of pieces of measurement data, and
the reference data acquisition unit is configured to determine to perform the data creation processing when a count of the plurality of pieces of measurement data stored in the storage unit is equal to or greater than a predetermined threshold value.

### Appendix 2

An in-vehicle device to be mounted in a vehicle, including:
a measurement information acquisition unit configured to acquire center-of-gravity information indicating a measurement result regarding a change in a center-of-gravity position of a driver of the vehicle;
a route information acquisition unit configured to acquire route information indicating a travel route of the vehicle, the travel route corresponding to the measurement result; and
a communication unit configured to transmit the measurement information and the route information to an external device outside the vehicle, wherein
the communication unit is configured to further transmit model information and driver identification information to the external device, the model information being information indicating a model of the vehicle, the driver identification information being information for identifying the driver.

### Appendix 3

A driver state determination device including:
processing circuitry,
the processing circuitry being configured to:
acquire a measurement result regarding a change in a center-of-gravity position of a driver of a vehicle;
acquire a travel route of the vehicle, the travel route corresponding to the acquired measurement result;
acquire reference data indicating a criterion for the change in the center-of-gravity position in a target route, the target route being the acquired travel route; and
perform determination processing of determining a state of the driver, based on the measurement result and the reference data.

### appendix 4

An in-vehicle device to be mounted in a vehicle, including:
processing circuitry,
the processing circuitry being configured to:
acquire measurement information indicating a measurement result regarding a change in a center-of-gravity position of a driver of the vehicle;
acquire route information indicating a travel route of the vehicle, the travel route corresponding to the measurement result; and
transmit the acquired measurement information and the acquired route information to an external device outside the vehicle.

### REFERENCE SIGNS LIST

1 vehicle
11 internal communication unit
12 measurement information acquisition unit
13 route information acquisition unit
14 external communication unit
15, 34 storage unit
21 pressure sensor
22 navigation device
31 communication unit
32 reference data acquisition unit
33 determination unit
51A, 51B CAN bus
101 in-vehicle device
151 external network
201 server
301 operation management device
501 driver state determination system
Tb1 correspondence table
Tb2 management table
R1, R2 route

1. A driver state determination device comprising:
a measurement result acquisition circuitry configured to acquire at least one measurement result regarding a change in a center-of-gravity position of a driver of a vehicle;
a travel route acquisition circuitry configured to acquire a travel route of the vehicle, the travel route corresponding to the measurement result;
a reference data acquisition circuitry configured to acquire reference data indicating a criterion for the change in the center-of-gravity position in a target route, the target route being the travel route; and
a determination circuitry configured to perform determination processing of determining a state of the driver, based on the measurement result and the reference data.

2. The driver state determination device according to claim 1, wherein
the measurement result acquisition circuitry is configured to acquire a target measurement result, the target measurement result being the measurement result in the target route, and
the reference data is created based on the target measurement result.

3. The driver state determination device according to claim 2, wherein the target measurement result is the measurement result for the driver to be subjected to the determination processing, and the reference data is created for each driver to be subjected to the determination processing.

4. The driver state determination device according to claim 2, wherein
the determination circuitry is configured to perform processing of determining whether an abnormality has occurred in the driver, based on the target measurement result,
the at least one measurement result includes a plurality of measurement results,
the measurement result acquisition circuitry is configured to acquire the plurality of measurement results including the target measurement result, and
the reference data is created based on the target measurement result and a measurement result based on which it is determined by the determination unit circuitry that no abnormality has occurred, among the plurality of measurement results.

5. The driver state determination device according to claim 1, wherein
the reference data acquisition circuitry is configured to create the reference data, based on the measurement result, and
the reference data acquisition circuitry is configured to exclude the measurement result from source data of the reference data when the measurement result satisfies a predetermined condition.

6. The driver state determination device according to claim 1, wherein the reference data indicates a criterion for the change in the center-of-gravity position in a case where a vehicle of an identical model to the vehicle travels along the target route.

7. The driver state determination device according to claim 1, further comprising:
a storage circuitry configured to store correspondence information indicating a correspondence relationship between the travel route and the reference data, wherein
the reference data acquisition circuitry is configured to acquire the correspondence information from the storage circuitry and acquire the reference data, based on the correspondence information.

8. An in-vehicle device to be mounted in a vehicle, comprising:
a measurement information acquisition circuitry configured to acquire measurement information indicating a measurement result regarding a change in a center-of-gravity position of a driver of the vehicle;
a route information acquisition circuitry configured to acquire route information indicating a travel route of the vehicle, the travel route corresponding to the measurement result; and
a communication circuitry configured to transmit the measurement information and the route information to an external device outside the vehicle.

9. A driver state determination system comprising:
an in-vehicle device to be mounted in a vehicle; and
a driver state determination device,
the in-vehicle device being configured to transmit measurement information and route information to the driver state determination device, the measurement information indicating a measurement result regarding a change in a center-of-gravity position of a driver of the vehicle, the route information indicating a travel route of the vehicle, the travel route corresponding to the measurement result,
the driver state determination device being configured to acquire reference data indicating a criterion for the change in the center-of-gravity position in the travel route indicated by the route information received from the in-vehicle device,
the driver state determination device being configured to determine a state of the driver, based on the measurement result indicated by the measurement information received from the in-vehicle device and the reference data.

10. A driver state determination method in a driver state determination device, the driver state determination method comprising:
acquiring a measurement result regarding a change in a center-of-gravity position of a driver of a vehicle;
acquiring a travel route of the vehicle, the travel route corresponding to the measurement result;
acquiring reference data indicating a criterion for the change in the center-of-gravity position in a target route, the target route being the travel route; and
performing determination processing of determining a state of the driver, based on the measurement result and the reference data.

11. A driver state determination method in an in-vehicle system including an in-vehicle device to be mounted in a vehicle and a driver state determination device, the driver state determination method comprising:
transmitting, by the in-vehicle device, measurement information and route information to the driver state determination device, the measurement information indicating a measurement result regarding a change in a center-of-gravity position of a driver of the vehicle, the route information indicating a travel route of the vehicle, the travel route corresponding to the measurement result;
acquiring, by the driver state determination device, reference data indicating a criterion for the change in the center-of-gravity position in the travel route indicated by the route information received from the in-vehicle device; and
determining, by the driver state determination device, a state of the driver, based on the measurement result indicated by the measurement information received from the in-vehicle device and the reference data.

12. A non-transitory storage medium storing driver state determination program to be used in a driver state determination device,
the driver state determination program causing a computer to function as:
a measurement result acquisition circuitry configured to acquire a measurement result regarding a change in a center-of-gravity position of a driver of a vehicle;
a travel route acquisition circuitry configured to acquire a travel route of the vehicle, the travel route corresponding to the measurement result;
a reference data acquisition circuitry configured to acquire reference data indicating a criterion for the change in the center-of-gravity position in a target route, the target route being the travel route; and
a determination circuitry configured to perform determination processing of determining a state of the driver, based on the measurement result and the reference data.

13. The driver state determination device according to claim 3, wherein
the determination circuitry is configured to perform processing of determining whether an abnormality has occurred in the driver, based on the target measurement result,
the at least one measurement result includes a plurality of measurement results,
the measurement result acquisition circuitry is configured to acquire the plurality of measurement results including the target measurement result, and
the reference data is created based on the target measurement result and a measurement result based on which it is determined by the determination circuitry that no abnormality has occurred, among the plurality of measurement results.

14. The driver state determination device according to claim 2, wherein
the reference data acquisition circuitry is configured to create the reference data, based on the measurement result, and
the reference data acquisition circuitry is configured to exclude the measurement result from source data of the reference data when the measurement result satisfies a predetermined condition.

15. The driver state determination device according to claim 3, wherein
the reference data acquisition circuitry is configured to create the reference data, based on the measurement result, and
the reference data acquisition circuitry is configured to exclude the measurement result from source data of the reference data when the measurement result satisfies a predetermined condition.

16. The driver state determination device according to claim 4, wherein
the reference data acquisition circuitry is configured to create the reference data, based on the measurement result, and
the reference data acquisition circuitry is configured to exclude the measurement result from source data of the reference data when the measurement result satisfies a predetermined condition.

17. The driver state determination device according to claim 2, wherein the reference data indicates a criterion for the change in the center-of-gravity position in a case where a vehicle of an identical model to the vehicle travels along the target route.

18. The driver state determination device according to claim 3, wherein the reference data indicates a criterion for the change in the center-of-gravity position in a case where a vehicle of an identical model to the vehicle travels along the target route.

19. The driver state determination device according to claim 4, wherein the reference data indicates a criterion for the change in the center-of-gravity position in a case where a vehicle of an identical model to the vehicle travels along the target route.

20. The driver state determination device according to claim 5, wherein the reference data indicates a criterion for the change in the center-of-gravity position in a case where a vehicle of an identical model to the vehicle travels along the target route.
